(19) United States
(12) Patent Application Publication
(10) Pub. No.: US 2023/0338627 A1
(43) Pub. Date: Oct. 26, 2023

(54) A SELF-CLEANING POROUS LAYER TO MINIMIZE THROMBUS FORMATION ON BLOOD CONTACTING DEVICES

(71) Applicant: UNIVERSITY OF PITTSBURGH - OF THE COMMONWEALTH SYSTEM OF HIGHER EDUCATION, PITTSBURGH, PA (US)

(72) Inventors: Jonathan VANDE GEEST, Gibsonia, PA (US); Ali BEHRANGZADE, Pittsburgh, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 18/008,476

(22) PCT Filed: Jun. 16, 2021
(86) PCT No.: PCT/US21/37582

§ 371 Date: Dec. 6, 2022

Related U.S. Application Data
(60) Provisional application No. 63/039,559, filed on Jun. 16, 2020.

(51) Int. Cl. A61L 33/00; A61L 27/50; A61L 27/56; A61L 27/34; A61L 33/06

(52) U.S. Cl. CPC A61L 33/0041; A61L 27/507; A61L 27/56; A61L 27/34; A61L 33/007; A61L 33/062; A61L 2420/02

(57) ABSTRACT

The invention relates to self-cleaning porous structures, e.g., layers or coatings, fabricated within, applied to, or deposited on a blood contacting surface of a medical device, to prevent activation and aggregation of platelets thereon. In certain embodiments, the layer or coating is composed of multi-layered fibers. The porous structure is applied to or deposited such as to form a permeable wall on the blood contacting surface. The blood travels into the wall and subsequently back out (reversing back into the lumen) during a cardiac cycle. Reversal flow is controlled during the diastole phase such that the backward flow repels the platelets and prevents their activation and aggregation and therefore, minimizes thrombus formation.

### CROSS-REFERENCE TO RELATED APPLICATIONS

This application claims priority under 35 U.S.C. § 119(e) from U.S. provisional patent application No. 63/039,559, entitled “A SELF-CLEANING POROUS LAYER TO MINIMIZE THROMBUS FORMATION ON BLOOD CONTACTING DEVICES” and filed on Jun. 16, 2020, the contents of which are incorporated herein by reference.

### FIELD OF THE INVENTION

The invention includes a self-cleaning porous layer or coating applied to or deposited on a blood contact medical device to minimize thrombus formation.

### BACKGROUND

Cardiovascular disease (CVD) has remained the leading cause of mortality in the United States and worldwide with an extremely high financial burden. Many surgical and interventional devices have been invented and utilized to treat CVD patients. However, most of these devices are still very susceptible to the formation of thrombosis due to contact between the foreign material and blood as well as disruption of the hemodynamic environment following these procedures. Formation of thrombosis has long been known as one of major causes of device failure in short-term (acute) and long-term (chronic) implantable medical devices. Device failure tremendously increases financial burden to the health care system while also jeopardizing patient safety. To address this critical clinical need, blood contacting devices such as vascular grafts, intravascular stents, and angioplasty equipment have been continuously evolving with a fast pace.

Surface topographical modification has been used to reduce the interaction between platelets and material and thus inhibit the formation of undesired blood clots. As an example, actuated surface wrinkles have been shown to reduce platelet activation. On the other hand, chemistry-based surface functionalization has been widely utilized to reduce thrombogenicity of cardiovascular blood contacting devices. For instance, heparin coating is used on vascular grafts and stents which can interfere with coagulation signaling pathways and thus reduce the formation of thrombosis. Since heparin is typically immobilized on the surface, this chain of chemical reactions becomes limited. The other limitation of using heparin is degradation upon exposure to biological environment which restricts its availability for the aforementioned reactions. Heparin can also detrimentally affect the coagulation cascade and lead to hemorrhage. Another widely-used method is polyethylene glycol (PEG) coating which increases the hydrophilicity of the surface and diminishes the protein adsorption to the surface of medical devices. The function of PEG can also be compromised during exposure to biological environment and thus the in-vivo results using PEG have been inconsistent. Among these blood contacting devices, vascular grafts are very susceptible to thrombus formation which ultimately leads to device failure.

According to the American Heart Association, approximately 6.5 million people aged 40 and older in the United States have Peripheral Arterial Disease (PAD). Also, coronary artery diseases (CAD) are known as the most prevalent cardiovascular disease in the United States. Atherosclerotic coronary arteries cause insufficient oxygenated nutrient-rich blood supply to the myocardium. This condition is called ischemia and ultimately results in myocardial infarction. These patients need intravascular stent implantation or arterial bypass surgery depending on the severity of the occlusion and patient's general health status. Autologous blood vessels such as saphenous vein and radial artery are still gold standards for coronary bypass while synthetic grafts are more typically used in peripheral vascular bypass. The availability of autologous tissues is limited due to pre-existing vascular disease, prior harvesting, and vein stripping. Furthermore, autologous grafts have a failure rate of 10-30% at 5 years depending on the graft type and synthetic grafts, such as ePTFE, also have high failure rate via intimal hyperplasia and graft thrombosis. These limitations necessitate the development of a vascular graft which is anti-thrombogenic while also possessing the desired mechanical properties. Inappropriate mechanical properties and thrombogenicity of biomaterials can disturb the homeostatic environment which will entail fatal consequences such as thrombosis and intimal hyperplasia.

Compliance-mismatch between the native artery and the adjacent vascular graft results in asynchronous expansion and recoil of the artery and graft thus altering blood flow patterns at the anastomotic sites which causes non-physiologic mechanical strain on the endothelial cells adjacent to the suture lines. Also, deviation of blood velocity profiles from normal conditions and the formation of stagnation areas lead to non-homeostatic shear stress which potentially activates platelets. These shear stress-activated platelets can lead to thrombosis at the anastomotic sites. In addition, non-physiological levels of shear stress is also the beginning of a series of undesired remodeling events including vessel stiffness, enhanced LDL metabolism, and rearrangement of the cell cytoskeleton.

Tissue engineered vascular grafts are made of synthetic and native biopolymers while synthetic grafts are mainly made of non-biodegradable materials including expanded polytetrafluoroethylene (ePTFE) and polyethylene terephthalate (Dacron). Interactions between blood components and these biomaterials can lead to protein adsorption, coagulation cascade activation, and platelet deposition.

The prior art techniques and approaches for treatment of CVD use chemical and surface topographical modifications which have limitations, including adverse effect on the patient and manufacturing challenges or limitations.

Thus, there is a need in the art to develop a means or mechanism to prevent activation and aggregation of platelets on the blood contacting surface of medical implants to reduce the formation of blood clots and device failure. The invention includes a fibrous coating on blood contacting surfaces which allows the blood to travel into the surface during systole and away from the surface during diastole, thus minimizing platelet adsorption and thrombogenesis.

### SUMMARY OF THE INVENTION

In one aspect, the invention provides a self-cleaning medical device that includes a blood contacting surface; a porous layer or coating fabricated within, applied to, or deposited on the blood contacting surface, comprising one or more materials selected from the group consisting of synthetic biodegradable materials, synthetic nonbiodegradable materials, hybrid biomaterials, and biopolymers, wherein one or more properties of the porous layer or coating are optimized to control reversal fluid flow there through.

The porous layer or coating can include multiple layers. The porous layer or coating can be a fibrous layer or a fibrous coating. The fibrous layer can include electrospun fibers. The electrospun fibers can be in the form of a fiber mat or web.

In certain embodiments, the synthetic biodegradable materials are selected from the group consisting of polyurethanes, PLA, PLGA, PGA, PCL, PLLA, gelatin, tropoelastin and mixtures and combinations thereof.

In certain embodiments, the synthetic nonbiodegradable materials are selected from the group consisting of silicones, polyurethanes, ePTFE, Dacron, and mixtures and combinations thereof.

In certain embodiments, the hybrid materials include one or more of the aforementioned synthetic biodegradable materials and one or more of the aforementioned synthetic nonbiodegradable materials.

In certain embodiments, the biopolymers comprise collagen, gelatin and tropoelastin. In certain other embodiments, the biopolymer is selected from the group consisting of fibrillary or nonfibrillar collagen, gelatin, tropoelastin, laminin, and mixtures and combinations thereof.

The one or more properties of the porous layer or coating can be selected from the group consisting of mechanical stiffness, material type, structural permeability and geometrical thickness.

The fibrous layer can be crosslinked with genipin or glutaraldehyde.

The medical device can be selected from the group consisting of a tissue-engineered vascular graft and a synthetic graft.

In another aspect, the invention provides a method of preparing a self-cleaning medical device. The method includes obtaining a medical device having a blood contacting surface; fabricating within, applying to, or depositing on the blood contacting surface a porous layer or coating comprising one or more materials selected from the group consisting of synthetic biodegradable materials, synthetic nonbiodegradable materials, hybrid biomaterials including at least one synthetic biodegradable material and at least one synthetic nonbiodegradable material and biopolymers; and pre-selecting one or more properties of the porous layer or coating to control reversal fluid flow there through.

In yet another aspect, the invention provides a method of reducing platelet activation and aggregation on a blood contacting surface of a medical device. The method includes obtaining a medical device having a blood contacting surface; fabricating within, applying to or depositing on the blood contacting surface a porous layer or coating comprising one or more materials selected from the group consisting of synthetic biodegradable materials, synthetic nonbiodegradable materials, hybrid biomaterials including at least one synthetic biodegradable material and at least one synthetic nonbiodegradable material and biopolymers; pre-selecting one or more properties of the porous layer or coating to control reversal fluid flow there through; and pushing platelets away from the blood contact surface to reduce platelet activation and aggregation on said surface.

### BRIEF DESCRIPTION OF THE DRAWINGS

A full understanding of the invention can be gained from the following description of the preferred embodiments when read in conjunction with the accompanying drawings, as follows:

FIG. 1 is a schematic that illustrates the mechanism of platelet repulsion during a cardiac cycle, in accordance with certain embodiments of the invention;

FIG. 2 is a plot that illustrates the results of an LDH assay to evaluate platelet deposition on a vascular graft surface with a soft inner layer or a stiff inner layer, in accordance with certain embodiments of the invention;

FIGS. 3A and 3B are SEM images that illustrate a soft inner layer surface of a vascular graft surface, in accordance with certain embodiments of the invention;

FIGS. 3C and 3D are SEM images that illustrate a soft inner layer surface of a vascular graft surface, in accordance with certain embodiments of the invention;

FIGS. 3E and 3F are SEM images that illustrate a stiff inner layer surface of a vascular graft surface, in accordance with certain embodiments of the invention;

FIGS. 3G and 3H are SEM images that illustrate a stiff inner layer surface of a vascular graft surface, in accordance with certain embodiments of the invention;

FIG. 4 shows the mechanical behavior of different biomaterials for fabrication of a porous vascular graft, in accordance with certain embodiments of the invention; and

FIG. 5 shows the tangent modulus of different biomaterials for fabrication of a porous vascular graft, in accordance with certain embodiments of the invention.

### DETAILED DESCRIPTION OF THE PREFERRED EMBODIMENTS

The invention relates to a self-cleaning porous structure, e.g., layer or coating, fabricated within, applied to, or deposited on a blood contacting surface of a medical device, e.g., that is exposed to pulsatile flow, to prevent activation and aggregation of platelets thereon. In certain embodiments, the layer or coating is composed of multi-layered fibers. The porous structure is fabricated within, applied to, or deposited such as to form a permeable layer or layers on the blood contacting surface with prescribed stiffness and permeability. The blood travels into the wall and subsequently back out (reversing back into the lumen) during a cardiac cycle. The porous structure allows blood to travel through the layer or coating during the systolic phase of the cardiac cycle and back during diastole phase. Reversal flow is controlled during the diastolic phase such that the backward flow repels platelets and prevents their activation and aggregation, and reduces blood-surface interaction. This phenomenon minimizes thrombus formation through two different mechanisms: (i) destabilizes the protein film that is formed on the surface almost immediately after the introduction of biomaterial; and (ii) pushes the platelets away from the surface using the fluid momentum during fluid flow reversal.

Furthermore, the present invention relates to novel, self-cleaning, anti-thrombotic synthetic surfaces. The inner surfaces of arteries and veins are naturally anti-thrombogenic, whereas synthetic materials placed in contact with blood quickly foul with thrombus. The synthetic surfaces according to the present invention include the porous structure fabricated within, applied to, or deposited on the blood contact surface to reduce or preclude thrombus fouling. When placed in contact with blood, for example, these synthetic surfaces having the porous structure fabricated within, applied thereto, or deposited thereon display significantly less platelet deposition and thrombosis as compared to the same materials exposed to blood and not having the porous structure applied thereto or deposited thereon.

In certain embodiments, wherein the medical device, e.g., vascular graft, is used to mimic a native vein or artery conduit, it is in the shape of a cylinder or tube. In this embodiment, the porous structure is fabricated within, applied to, or deposited on the inner layer of the cylinder or tube (i.e., forms the surface of the inner cavity wherein blood flows through). Target applications include, but are not limited to, below knee lower extremity bypass and coronary bypass (CABG) operations, hemodialysis grafts, as well as venous reconstruction.

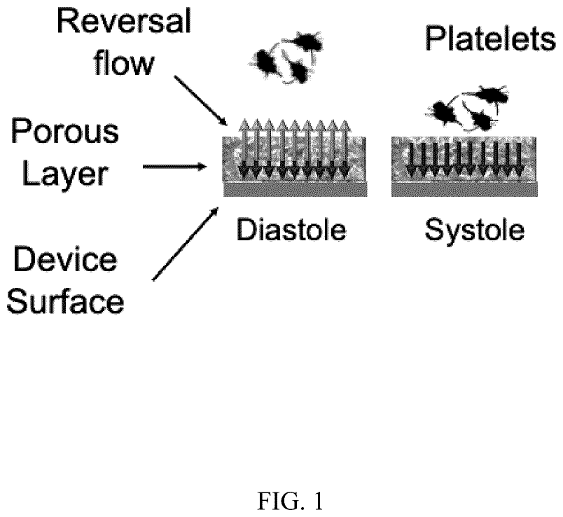

FIG. 1 illustrates the mechanism of platelet repulsion for a medical device, which shows a device surface 1 that is a blood contacting surface of the medical device, a porous layer 2 fabricated within, applied to, or deposited on the device surface 1 (blood contacting surface), a direction of blood flow during systole 3 (the systolic phase), and a reversal flow 4 during diastole 5 (the diastolic phase) such that the backward flow repels platelets 6. The characteristics of the reversal flow 4 are controlled by manipulation of the porous structure properties, i.e., mechanical (stiffness), structural (permeability) and geometrical (thickness). The porous layer 2 (e.g., fibrous porous layer) is multi-layered and fabricated within or deposited on the device surface 1 (blood contacting surface of the medical device) by various manufacturing methods, while the permeability is modulated through a various methods including laser cutting, spray phase separation, thermally induced phase separation (TIPS), porogen leaching, and deposition with subsequent removal using sacrificial materials. This mechanism has potential applications in any blood contacting devices that are exposed to a pulsatile flow. Non-limiting suitable applications include but are not limited to vascular grafts which expand and recoil in response to the pulsatile flow of the cardiovascular system, as well as balloon catheters and cannula which are used in the vascular system. Generally, blood contacting surfaces of these medical devices are exposed to pulsatile flow and prone to thrombus formation.

During the systolic phase of a cardiac cycle, the vascular graft expands and fluid, e.g., blood, travels into the graft wall and away from the lumen. During the diastolic phase, the flow is reversed (Transmural Reversal Flow, TRF) if the vascular graft has particular geometric and material (stiffness, permeability) properties. Vascular grafts according to the invention have a porous structure, and blood flow can travel through this porous medium as the intraluminal pressure changes in the systolic and diastolic phases. This is similar to interstitial fluid flow through the extracellular matrix (ECM) during a cardiac cycle. The transmural fluid flow in a graft can be reversed during a cardiac cycle and moved back into the lumen. The fluid behavior in this phenomenon greatly depends on the mechanical, structural and geometrical properties of different layers of the vascular graft. Without intending to be bound by any particular theory, it is believed that the reversal flow can repel the platelets from the inner layer before they can firmly attach thereto.

Suitable porous materials for use in the invention include synthetic biodegradable materials, such as but not limited to polylactic acid (PLA), poly(lactic-co-glycolic acid) (PLGA), poly(glycolic acid) (PGA), polycaprolactone (PCL), poly-L-lactic acid (PLLA), poly(vinyl alcohol) (PVA), and polyurethanes; synthetic nonbiodegradable materials such as but not limited to silicones, expanded polytetrafluoroethylene (ePTFE), polyethylene terephthalate (Dacron) and polyurethanes; hybrid biomaterials combining synthetic biodegradable and synthetic nonbiodegradable materials, and biopolymers such as but not limited to collagens—fibrillar and nonfibrillar, tropoelastin, and laminin; and mixtures and combinations thereof. Combinations (of all ratios) of synthetic polymers and native biopolymers are considered for use in the invention. In certain embodiments, a combination of polyurethanes selected from tecoflex, tecothane, carbothane and other medical polymers from Lubrizol Advanced Materials, and Dacron material are used as porous materials in the invention.

The porous material is used to form the multi-layer fibrous layer or coating that is fabricated within, applied to, or deposited on the blood contacting surface of the medical device, e.g., vascular graft. There are various apparatus and techniques known in the art for preparing the fibers that compose the multi-layer fibrous layer. In certain embodiments, the fibers include but are not limited to microfibers and nanofibers. In certain embodiments, the fibers are formed by electrospinning. The electro-spinning process is typically carried out at certain controllable temperature and humidity conditions. The electro-spinning apparatus includes a syringe pump and a conductive substrate. The syringe pump includes a syringe containing an electro-spinnable solution, such as a polymer or combination of polymer components dissolved in solvent, e.g., a polymer molten mass. A capillary is located at the tip of a dispenser, which is coupled with the pole of a voltage-generating arrangement (current supply). By means of an injection pump, the solution is transported out of the syringe via tubing towards the spinning capillary, where drops are formed at the tip. The surface tension of the drop of the solution coming out of the spinning capillary is overcome by means of an electric field between the spinning capillary and a counter electrode. The drop injected by the syringe coming out of the spinning capillary deforms and, when it reaches a critical electric potential, it is drawn to yield a fine filament (a so-called jet). This electrically-charged jet, continuously extracting new solution from the spinning capillary, is then accelerated in the electric field towards the counter electrode. The jet solidifies during its flight towards the counter electrode by means of the evaporation of the solvent or by means of cooling, such that in a short period of time continuous nanofibers are generated, linked with one another, with typical diameters of a few nanometers to several micrometers. The nanofibers are collected on the conductive substrate. In other embodiments, layers with controlled permeability are created by thermally induced phase separation (TIPS), porogen leaching, spray phase separation, solvent casting, and single or two photon laser cutting.

In certain embodiments, the nanofibers are deposited on a rotating drum that includes a roller, to form a nanofiber mat or web. The conductive substrate is positioned onto the roller, such that the substrate rotates thereon. The use of a rotating drum (roller) that is conductive does not need any other conductive substrate. The conductive substrate is selected from materials known in the art, such as, but not limited to stainless steel and aluminum. The syringe pump deposits the solution onto the conductive substrate as the roller is rotated. The rotating, conductive substrate serves as a collector and is grounded. The dispenser has translational movement to modulate fiber orientation if desired. The conductive substrate is removed from the rotating drum to form a nanofiber mat, and the nanofiber mat is applied to the blood contacting surface of the medical device, e.g., vascular graft.

Suitable lengths for the nanofibers range from, as short as, a few millimeters to, as long as, several feet, as desired and dictated by the volume of the spinning solution and the applied potential bias forming a flexible and pliable form. The nanofiber surfaces that are produced exhibit a smooth or irregular surface topography depending on the nature of the bias and the viscosity of the spinning solution.

The polymer component is selected from a variety of known polymers as aforementioned herein. The polymer component can further or additionally include other known organic, inorganic or metal materials, and mixtures thereof.

The solvent is selected from known solvents, such as, but not limited to, 1,1,1,3,3,3-hexafluoro-2-propanol (HFP), ethanol, chloroform, DI water, tetrahydrofuran (THF), dichloromethane (DCM), dimethyl formamide (DHF), trifluoroacetic acid, and mixtures and combinations thereof.

As aforementioned, in certain embodiments, the electro-spun fibers are interconnected to form a nanofiber/microfiber web or mat. The diameters of the fibers vary and in certain embodiments, are from about 10 nanometers to about 100 microns. In certain embodiments, the electro-spun fibers are from 1-7 μm. The individual nanofibers in the mat, e.g., nonwoven mat, have a random orientation or are predominantly oriented in one or more directions.

In certain embodiments the fibrous coating or layer, e.g., nanofiber mat or web, is crosslinked with a crosslinker, such as but not limited to 0.5% genipin in ethanol or glutaraldehyde vapor.

In accordance with certain embodiments of the invention, electro-spun nano-fiber composites, e.g., mats, can be prepared as follows:
i) Dissolving polymer in solvent to form a solution; and
ii) Electro-spinning the solution into a plurality of nano-fibers, e.g., in the form of a mat, typically, having diameters of a few nanometers to several micrometers (e.g., in certain embodiments, from greater than about 100 nanometers to about 10 μm) and lengths, as short as, a few millimeters to, as long as, several feet, as desired; and dependent on the volume of the spinning solution and the applied voltage, flow rate of syringe pump, and viscosity of the spinning solution; and applying to or depositing on the blood contacting surface of the medical device.

The medical device is composed of multilayered porous materials whose stiffness, permeability, and thickness are optimized to maximize reversal flow (flow back into the lumen of the artery). The stiffness of a material is the degree to which it can deform in response to an imposed load, which in this case is the cyclic intraluminal pressure in the artery. Manipulation of stiffness can be achieved by methods including but not limited to varying the stiffness of the nonporous material that is being electrospun or fabricated, varying the of time and/or concentration of chemical crosslinkers, physical intermingling of nano/micro fibers during electrospinning, controlling the orientation of fibers during electrospinning, altering the parameters controlling sacrificial material removal, altering the size and shape of porogens when using porogen leaching, controlling the size/distribution of water and polymer droplets in spray phase separation, and manipulating TIPS parameters. Stiffness of the porous materials are characterized by using methods including but not limited to uniaxial testing, biaxial testing, pressure inflation testing, and confined/unconfined compression testing. Given the nonlinear nature of synthetic and biopolymer materials, the mechanical response (from which stiffness is calculated) of these materials is assessed under large deformations. Stiffness is typically quantified as the local tangent slope of the curve that represents the mechanical response. Typical values for some but not all suitable polymers for use in the invention are included in the Examples herein. Note that parameters (e.g., C₁₀, C₂₀, and the like) that describe the nonlinear behavior of these materials is needed, and not a linear elastic Young's Modulus. The thickness of the layers are easily controlled by altering electrospinning settings (e.g., flow rate, voltage, distance, drum rotation/axial speed, duration of spinning) or parameters used in other fabrication approaches. The permeability of the layers are controlled using an approach including but not limited to controlling some or all of the parameters that control electrospinning, porogen leaching, TIPS, laser cutting, and spray phase separation.

In accordance with the invention, the characteristics of the reversal flow is controlled by manipulation of the porous structure properties, i.e., mechanical (stiffness), structural (permeability) and geometrical (thickness). Thus, the fibrous layer or coating as formed by the electrospun nanofibers is prepared to exhibit various desired properties. In certain embodiments, the fibrous layer or coating provides a soft inner layer for the blood contacting surface of the medical device, e.g., vascular graft, and in certain other embodiments, the fibrous layer or coating provides a stiff inner layer for the blood contacting surface of the medical device, e.g., vascular graft. Further, the permeability, thickness, and stiffness of the layers of the device are adjusted to provide a the desired response in the medical device, e.g., vascular graft.

In certain embodiments of the invention, the medical device is a tissue-engineered vascular graft (TEVG). Tissue engineered vascular grafts are made of synthetic and native polymers including but not limited to polycaprolactone (PCL), polylactic acid (PLA), tropoelastin, gelatin, and collagen, while synthetic grafts are mainly made of non-biodegradable materials including expanded polytetrafluoroethylene (ePTFE) and polyethylene terephthalate (Dacron). Interactions between blood components and these biomaterials lead to protein adsorption, coagulation cascade activation, and platelet deposition. Application or deposition of the self-cleaning porous structure, e.g., layer or coating, on the blood contacting surface of the vascular graft prevents activation and aggregation of platelets thereon. As aforementioned, the blood travels into the permeable wall of the porous structure during the systolic phase of the cardiac cycle and back during diastole phase to prevent platelet activation and aggregation, and minimize thrombus formation on the blood contacting surface.

In certain embodiments, the medical device of the invention is a medical implant device, e.g., vascular graft. The medical implant device is implanted into a body, e.g., human body, of a patient.

The invention includes one or more of the following advantages: (i) an optimized porous layer that has no interference of the coagulation cascade and thus, no hemorrhage; (ii) no polymer denaturization in exposure to in-vivo environment; and no chemical components involved and thus, no challenge in surface binding; (iii) flexibility to utilize different hemocompatible materials and further contribute to reducing the platelet activation; (iv) use of the blood pressure as the actuation force for the fluid movement through the porous medium; and (v) reducing the interaction of blood and surface.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

### EXAMPLE

The mechanism of platelet repulsion for a medical device illustrated in FIG. 1 was computationally investigated in a bi-layered vascular graft. A two-dimensional axisymmetric porohyperelastic finite element model was developed in ABAQUS. The following equation demonstrates the strain energy potential function for a polynomial material model.

$$U = {\sum }_{i + j = 1}^{N} {{C}_{ij} ( {\overline{I}}_{1} - 3 )}^{i} {( {\overline{I}}_{2} - 3 )}^{j} + {\sum }_{i = 1}^{N} \frac{1}{{D}_{i}} {( {J}^{e ℓ} - 1 )}^{2 i}$$

wherein U is the strain energy per unit of reference volume, N is a material parameter (e.g., N=2 for second order polynomial), Cᵢⱼ and D₁ are material parameters, and Ī₁ and Ī₂ are the first and second deviatoric strain invariants. In this 2D model, each layer has three different mechanical properties, C₁₀, C₂₀, D₁ and one structural property called permeability (k). In addition, the thickness of each layer affects the mechanics and fluid flow. In total, eight properties were assigned to the graft: inner layer: C₁₀ⁱ, C₂₀ⁱ, D₁, kⁱ; outer layer: C₁₀^{o}, C₂₀^{o}, D₁, k^{o}; inner layer thickness (outer layer constitutes the rest of the graft wall). The values of D are the same for both layers.

Fluid transport in a porous medium in ABAQUS is governed by Darcy's law:

$${f}_{i} = - \frac{{k}^{f {f}_{A}}}{{γ}_{ω}} ( \frac{\partial {p}^{f}}{\partial {x}_{i}} )$$

wherein fᵢ is the volumetric flow rate per unit area, k^{ff}_{A} is the permeability which is calculated by:

k^{ff}_{A}=k^{ff}*g*p

permeability is considered isotropic and the apparent relative fluid velocity of the permeating fluid is calculated as follows:

vᵢ^{fr}=n(vᵢ^{f}−vᵢ)

wherein n is the porosity, vᵢ^{f} is the velocity of the fluid in the entire continuum, and vᵢ is the velocity of the solid. Simulations were implemented for different combinations of inner to outer layer thickness ratios, permeability and stiffness for each layer. A cyclic intraluminal pressure (400 cycles) in the range of 80 to 120 mmHg was applied (diastolic and systolic pressure) and the peak relative fluid velocity was considered for optimization. The ratio of relative fluid velocity was considered as follows,

$$Ratio of relative fluid velocity = \frac{{\sum }_{step = 1}^{100} {V}_{- x} . Δ t}{{\sum }_{step = 1}^{100} {V}_{+ x} . Δ t}$$

wherein V₋ₓ, and V₊ₓ, are velocities in −x and +x direction, respectively and Δt=0.015 s is the time step. This fraction delineates how far the fluid was pushed back relative to how far it traveled through the porous medium. The relative fluid velocities were considered at the first node. The total thickness of the graft was 100 μm, and element size was determined to be 0.5 μm using mesh convergence study.

The range of effect of all simulation parameters was also investigated. All parameters were shown to influence the reversal flow. However, stiffness and thickness ratio provided more controllability of the flow kinetics. There is an ability to control these parameters during manufacturing process to a larger extent as compared to the range of parameters that were restricted in sensitivity study and testing.

For optimization, a cyclic pressure pattern consistent with rat aortic pressure was applied to the lumen and pressure cycling continued until a steady state was reached in each simulation. The steady state criterion was defined based on a specific percentage of a platelet length and the clotting time of a rat.

In order to optimize the eight parameters, an objective function was defined to simultaneously match the in-vivo compliance of the vascular graft to a target artery while maximizing TRF. In this objective function, the Euclidean distance between the calculated compliance and TRF and a target value was calculated, while normalizing both terms. Although, this function is subject to change as different weights (ω₁, ω₂) may be assigned, and it also can have different forms.

$$f = \sqrt{[ {( {ω}_{1} \frac{{f}_{1} - {f}_{1}^{( indiv - opt )}}{{f}_{1}^{( indiv - opt )}} )}^{2} + {( {ω}_{2} \frac{{f}_{2} - {f}_{2}^{( indiv - opt )}}{{f}_{2}^{( indiv - opt )}} )}^{2} ]}$$

wherein,
f₁=computed compliance and f₂=e^{vfr};
The TRF (f₂) is the exponential transformation of fluid velocity;
f₂^{indiv-opt} is the individually optimized reversal flow generated from a pre-optimization (in this pre-optimization, only reversal flow was considered—no compliance is involved);
Rat aorta was chosen as the target vessel;
Peak TRF was considered for optimization;
Weights were assigned equally (ω₁=0.01 and ω₂=0.99)— this could vary.

All the optimizations were done by coupling MATLAB and ABAQUS.

This is an example of an approach to optimize the layered graft for rat aorta. This can be changed based on the target vessel compliance and geometry.

### Pre-Optimization

A sequential (two-step) particle swarm optimization algorithm was utilized to optimize the parameters in order to achieve the maximum possible reversal flow in a bi-layered graft while also minimizing the probability of attaining a local minimum. There were 240 particles used to explore the entire domain appropriately to find the global (not local) minimum of the objective function. A very wide range was considered for each optimization parameter to explore all possibilities. In the first step of optimization, we started with no initial guess and the result of this step was used as an initial guess for the second step to make sure the local minimum is found. The following table shows the optimized values. The peak TFR was approximately 312 μm/s. Note that the length of a single platelet is ˜2-3 μm.

| Inner layer | | | | | Outer layer | | | | |
|---|---|---|---|---|---|---|---|---|---|
| C₁₀ | C₂₀ | | K | Thickness | C₁₀ | C₂₀ | | K | Thickness |
| (kpa) | (kpa) | D₁ | (m/s) | (μm) | (kpa) | (kpa) | D₁ | (m/s) | (μm) |
| 57.8 | 30 | 1.53e−5 | 6.23e−4 | 90 | 30 | 30 | 1.53e−5 | 3.19e−11 | 10 |

### Main Optimization

A sequential (two-step) particle swarm optimization algorithm was utilized to optimize the parameters in order to achieve the maximum possible reversal flow and match the compliance of a bi-layered graft to rat aorta. There were 240 particles used to explore the entire domain and a very wide range was considered for each optimization parameter (same as pre-optimization). In the first step of optimization, we started with no initial guess and the result of this step was used as an initial guess for the second step to make sure the local minimum is found. The following table shows the optimized values. The peak TFR was approximately 144 μm/s and the compliance was matched to rat aortic compliance with <1% error.

| Inner layer | | | | | Outer layer | | | | |
|---|---|---|---|---|---|---|---|---|---|
| C₁₀ | C₂₀ | | K | Thickness | C₁₀ | C₂₀ | | K | Thickness |
| (kpa) | (kpa) | D₁ | (m/s) | (μm) | (kpa) | (kpa) | D₁ | (m/s) | (μm) |
| 30 | 1.13e6 | 1.53e−5 | 3.2e−4 | 90 | 30 | 4.87e4 | 1.53e−5 | 9.3e−12 | 10 |

### Final Consideration

In the optimized graft, there was a possibility that the graft was ‘leaking’ too much. However, it was considered that a trilayered design with a very thin outer layer being almost impermeable would potentially prevent this phenomenon and also provide excellent suturability.

Different polymers were tested in a uniaxial tensile testing device to determine potential candidate for fabrication of each layer. All grafts were made using electrospinning technique. The solution concentrations varied from 8 to 20% for different polymers, and different solvents were used for different polymers. 1 mL of solution was spun for all the grafts, and a majority of the materials were spun with a flow rate of 0.05 ml/min. A detailed description of electrospinning condition for each material can be found below. A strip was cut from the tubular graft axially and used for uniaxial tensile testing (N=5 each). All samples except PVA were hydrated during the test. Gelatin constructs were made and crosslinked with 0.5% and 2.5% genipin in ethanol at 37 c for 24 hours. Strips of materials were hooked to the clippers of a uniaxial tensile testing device and stretch up to 15% of original length with 0.1 mm/s speed. Force and length were recorded, and Cauchy stress was plotted against stretch ratio.

| | conc. | | | Flow rate | Working |
|---|---|---|---|---|---|
| Material | (% w/v) | solvent | Voltage | (ml/min) | distance |
| PLA | 12 | HFP | 15 | 0.05 | 10 |
| PCL | 10 | HFP | 15 | 0.05 | 10 |
| PVA | 10 | DI water | 25 | 0.01 | 10 |
| PLGA | 20 | HFP | 15 | 0.05 | 10 |
| PU | 8 | HFP | 15 | 0.05 | 10 |
| tecoflex | 8 | HFP | 15 | 0.05 | 10 |
| gelatin 0.5% | 10 | HFP | 15 | 0.05 | 10 |
| gelatin 2.5% | 10 | HFP | 15 | 0.05 | 10 |
| Polyethylene | 10 | TFA | 10 | 0.02 | 12 |
| Polystyrene | 25 | DMF | 15 | 0.016 | 10 |

The effect of inner layer material stiffness (not optimized) on platelet adhesion was tested in one experiment (n=1). For this purpose, two bi-layered grafts were electrospun and crosslinked. The outer layer of both grafts was made of pure PCL and thus had the same stiffness. The inner layer of both grafts had the same amount of gelatin/PCL ratio to have the same material in both grafts' blood contact surface. The inner layer of one graft was crosslinked with 0.5% genipin in ethanol (soft inner layer) and the other graft was crosslinked with 5% genipin in ethanol (stiff inner layer). Both of these grafts were used in a flow loop. In this flow loop, the grafts were cannulated using capillaries and sutured/glued to ensure no leakage from ends. The capillaries were then connected to tubing and fresh ovine blood was circulated for 120 minutes at 37 c. A peristaltic pump (pulsatile flow) circulated the blood in the system including the grafts. At the end, the circuit was gently washed using PBS and constructs were stored in PBS for LDH assay and SEM imaging. For LDH assay, the constructs were rinsed with PBS 10 times and then immersed in 2% Triton X and stirred for 20 minutes. The vial containing the solution and construct were centrifuged at 250×g for 10 minutes. Simultaneously, ovine blood was centrifuged at 250× for 20 minutes to get the platelet rich plasma. Then the supernatant was exposed to 2% Triton X and stirred for 20 minutes. These samples were also centrifuged at 250×g for 10 minutes. Constructs and controls (platelet rich plasma from ovine blood) were incubated for 30 minutes at 4 c. Absorbance of all samples were measured using a spectrophotometer.

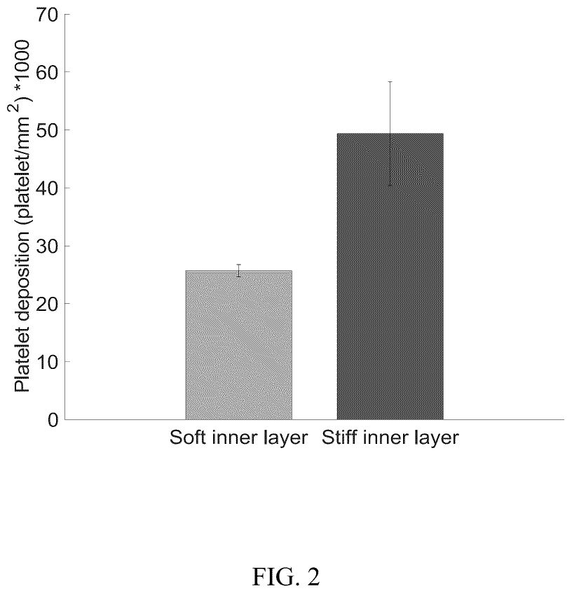

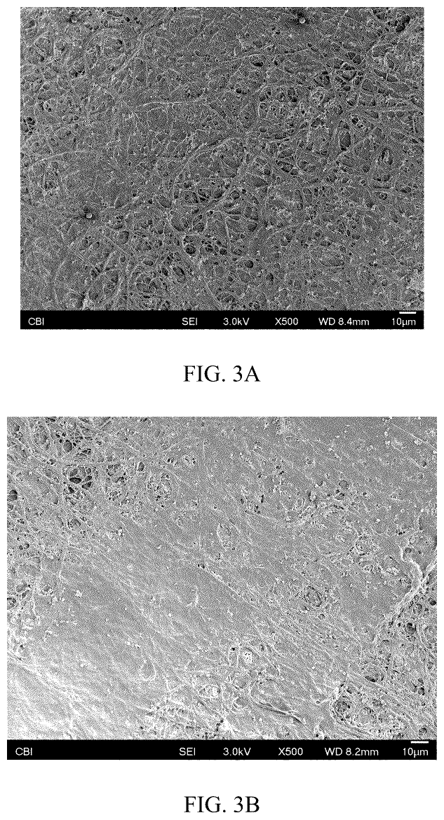

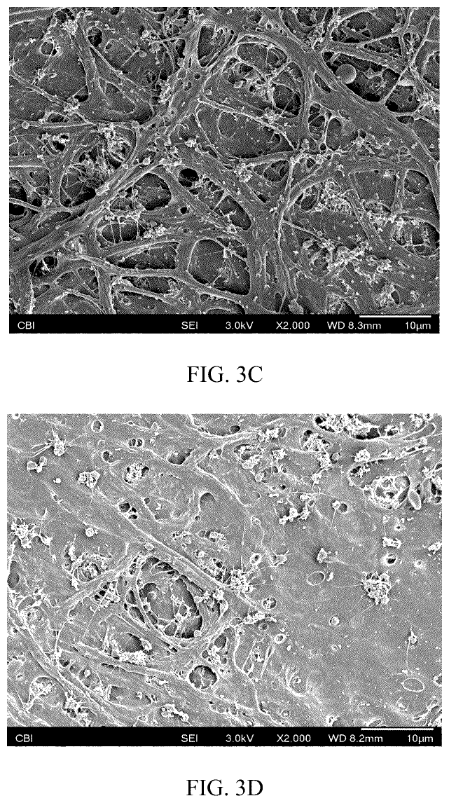

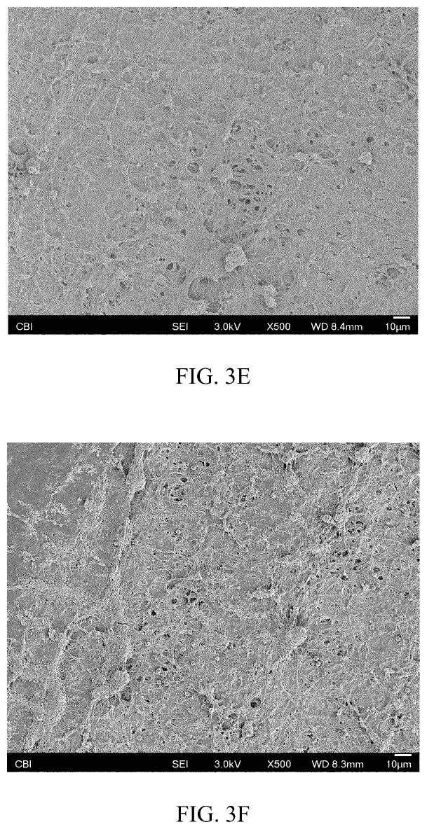

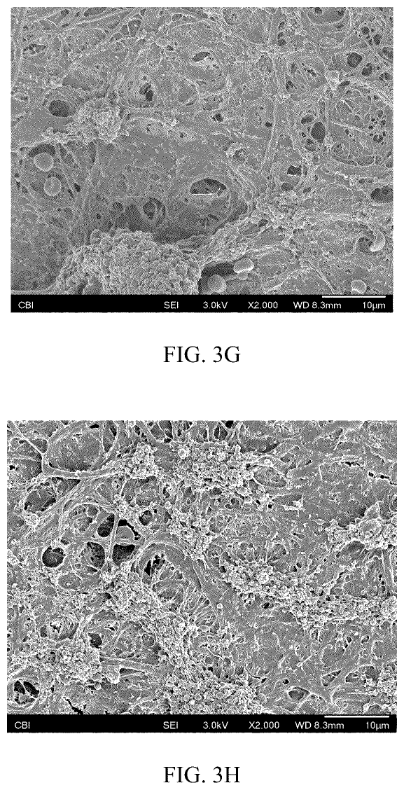

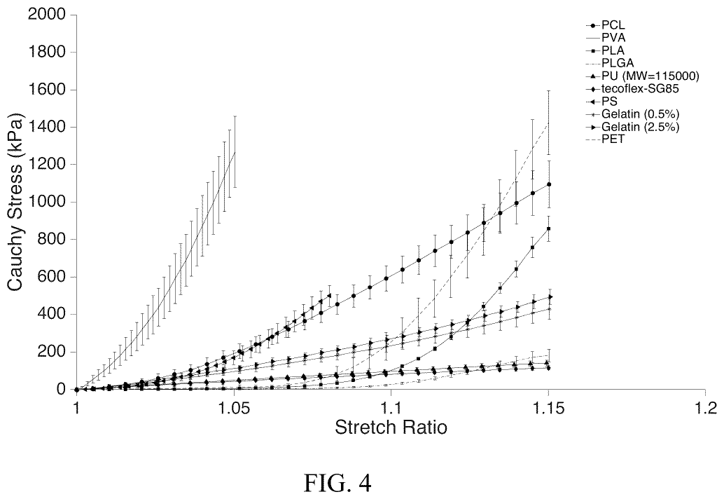

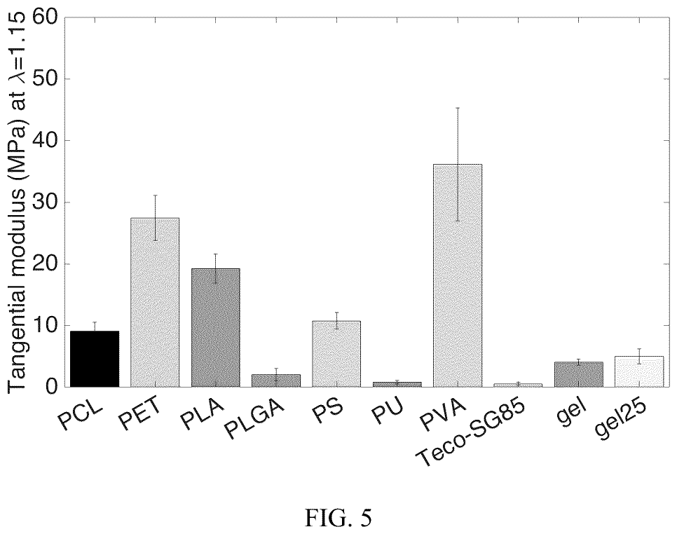

FIG. 2 shows the results of the LDH assay. The platelet deposition on the graft surface having the soft inner layer was much less as compared to the stiff inner layer. FIGS. 3A-H show SEM images at 500× and 200× for the soft inner layer or stiff inner layer, which were taken to confirm the LDH results. Both of the LDH assay results and SEM images confirmed that there was much less platelet deposition on the graft with the soft inner layer on the blood contacting surface. FIG. 4 shows the mechanical behavior of different biomaterials for fabrication of a porous vascular graft, and FIG. 5 shows the tangent modulus of different biomaterials for fabrication of a porous vascular graft.

We claim:

1. A self-cleaning medical device, comprising:
a blood contacting surface;
a porous layer or coating fabricated within, applied to, or deposited on the blood contacting surface, comprising one or more materials selected from the group consisting of synthetic biodegradable materials, synthetic nonbiodegradable materials, hybrid biomaterials, and biopolymers,
wherein one or more properties of the porous layer or coating are optimized to control reversal fluid flow there through.

2. The medical device of claim 1, wherein the porous layer or coating comprises multiple layers.

3. The medical device of claim 1, wherein the porous layer or coating is a fibrous layer or a fibrous coating.

4. The medical device of claim 3, wherein the fibrous layer comprise electrospun fibers.

5. The medical device of claim 4, wherein the electrospun fibers are in the form of a fiber mat or web.

6. The medical device of claim 1, wherein the synthetic biodegradable materials are selected from the group consisting of polyurethanes, PLA, PLGA, PGA, PCL, PLLA, gelatin, tropoelastin and mixtures and combinations thereof.

7. The medical device of claim 1, wherein the synthetic nonbiodegradable materials are selected from the group consisting of silicones, polyurethanes, ePTFE, Dacron, and mixtures and combinations thereof.

8. The medical device of claim 1, wherein the hybrid materials include one or more of the synthetic biodegradable material as recited in claim 6 and one or more of the synthetic nonbiodegradable material as recited in claim 7.

9. The medical device of claim 1, wherein the biopolymers comprise collagen, gelatin and tropoelastin.

10. The medical device of claim 7, wherein the biopolymer is selected from the group consisting of fibrillary or nonfibrillar collagen, gelatin, tropoelastin, laminin, and mixtures and combinations thereof.

11. The medical device of claim 1, wherein the one or more properties are selected from the group consisting of mechanical stiffness, material type, structural permeability and geometrical thickness.

12. The medical device of claim 3, wherein the fibrous layer is crosslinked with genipin or glutaraldehyde.

13. The medical device of claim 1, wherein said device is selected from the group consisting of a tissue-engineered vascular graft and a synthetic graft.

14. A method of preparing a self-cleaning medical device, comprising:
obtaining a medical device having a blood contacting surface;
fabricating within, applying to, or depositing on the blood contacting surface a porous layer or coating comprising one or more materials selected from the group consisting of synthetic biodegradable materials, synthetic nonbiodegradable materials, hybrid biomaterials including at least one synthetic biodegradable material and at least one synthetic nonbiodegradable material and biopolymers; and
pre-selecting one or more properties of the porous layer or coating to control reversal fluid flow there through.

15. A method of reducing platelet activation and aggregation on a blood contacting surface of a medical device, comprising:
obtaining a medical device having a blood contacting surface;
fabricating within, applying to, or depositing on the blood contacting surface a porous layer or coating comprising one or more materials selected from the group consisting of synthetic biodegradable materials, synthetic nonbiodegradable materials, hybrid biomaterials including at least one synthetic biodegradable material and at least one synthetic nonbiodegradable material and biopolymers;
pre-selecting one or more properties of the porous layer or coating to control reversal fluid flow there through; and
pushing platelets away from the blood contact surface to reduce platelet activation and aggregation on said surface.
